(19) United States
(12) Patent Application Publication
(10) Pub. No.: US 2019/0001097 A1
(43) Pub. Date: Jan. 3, 2019

(54) MULTI-FUNCTIONAL MASKING ASSEMBLY WITH HUMIDITY CONTROL

(71) Applicant: Tong Hoon SOHN, Yongin-si, Gyeonggi-do (KR)

(72) Inventor: Tong Hoon SOHN, Yongin-si, Gyeonggi-do (KR)

(21) Appl. No.: 15/748,682

(22) PCT Filed: Nov. 14, 2016
(86) PCT No.: PCT/KR2016/013108

§ 371 Date: Jan. 30, 2018

(30) Foreign Application Priority Data
Nov. 27, 2015 (KR) 10-2015-0167812

(51) Int. Cl. A61M 16/16; A41D 13/11; A61M 16/06; A61M 16/10; A61M 16/14

(52) U.S. Cl. CPC A61M 16/16; A41D 13/1161; A61M 16/06; A61M 2205/0266; A61M 16/142; A41D 2400/32; A61M 16/105

(57) ABSTRACT

The present invention provides a multi-functional masking assembly having a humidity adjusting function, and having a sheet module provided on the inner side surface of the mask worn to cover the nose and the mouth of the face, wherein the sheet module is located on the cheek parts of the face, and water is supplied to the sheet module so as to supply humidity to the nose and the mouth of the face and to adjust humidity around the nose and the mouth while the mask is worn, thereby: preventing a cold, dryness due to the air during air conditioning, and inhalation of polluted air, yellow dust, fine dust, and the like caused by air pollution while increasing respiratory health; and enabling people of all ages and genders to easily use the mask. The multi-functional masking assembly with humidity control includes a mask (10) having loops (11) provided on both ends thereof and hung on the ears of an user, or female and male coupler members (12a, 12b) provided on both ends of the mask, wherein the female and male coupler members (12a, 12b) are removably coupled to each other; a sheet module (20) located on cheek portions of an inner face of the mask (10); and a water supply member (30) configured for supplying water to the sheet module (20).

### TECHNICAL FIELD

The present disclosure relates to a multi-functional masking assembly with humidity control. More particularly, the present invention relates to a multi-functional masking assembly having a humidity adjusting function, and having a sheet module provided on the inner side surface of the mask worn to cover the nose and the mouth of the face, wherein the sheet module is located on the cheek parts of the face, and water is supplied to the sheet module so as to supply humidity to the nose and the mouth of the face and to adjust humidity around the nose and the mouth while the mask is worn, thereby: preventing a cold, dryness due to the air during air conditioning, and inhalation of polluted air, yellow dust, fine dust, and the like caused by air pollution while increasing respiratory health; and enabling people of all ages and genders to easily use the mask.

### RELATED ART

As noted, the masking device prevents inhalation and direct contact of cold and dry air. It prevents foreign objects such as dust from being sucked into the respiratory tract.

The conventional masking device is composed of a masking assembly body made of cloth having a size enough to cover a user's mouth and nose, and strings mounted on both sides of the body so as to hang on the wearer's ear.

Although such a masking device is used to prevent dust inhalation and to resist cold air, the masking assembly has a disadvantage that it cannot perform the function of suppressing dryness of the respiratory system by supplying moisture.

Further, respiratory diseases due to contaminants such as dust and fine dust and dry air may not be prevented by the masking device.

### SUMMARY OF THE INVENTION

### Technical Purpose

The present invention is provided to solve the above problems. More particularly, the present invention provides a multi-functional masking assembly having a humidity adjusting function, and having a sheet module provided on the inner side surface of the mask worn to cover the nose and the mouth of the face, wherein the sheet module is located on the cheek parts of the face, and water is supplied to the sheet module so as to supply humidity to the nose and the mouth of the face and to adjust humidity around the nose and the mouth while the mask is worn, thereby: preventing a cold, dryness due to the air during air conditioning, and inhalation of polluted air, yellow dust, fine dust, and the like caused by air pollution while increasing respiratory health; and enabling people of all ages and genders to easily use the mask.

### Technical Solutions

In a first aspect, there is provided a multi-functional masking assembly with humidity control, the assembly comprising: a mask (10) having loops (11) provided on both ends thereof and hung on the ears, or female and male coupler members (12a, 12b) provided on both ends thereof; a sheet module (20) located on cheek portions of an inner face of the mask (10) and provided on the inner face of the mask (10); and a water supply member (30) configured for supplying water to the sheet module (20).

In one embodiment, the sheet module (20) is composed of cotton, charcoal, and sponge.

In one embodiment, the water supply member (30) includes a water supply channel (31e), wherein the water supply channel (31e) include a vertical channel (31e-1), a cylindrical channel (31e-2) and a communication channel (31e-3) defined for connecting the vertical channel (31e-1) and the cylindrical channel (31e-2) to each other, wherein a lower end of the sheet module (20) is inserted into an inner space (31b) defined in the vertical channel (31e-1) through an through-hole 31a defined at a top portion of the vertical channel (31e-1), wherein external water is injected into the cylindrical channel (31e-2) through an injection hole (31c) defined at the cylindrical channel (31e-2), wherein the injected water is transferred into the vertical channel (31e-1) through the communication channel (31e-3), wherein a plug (31d) is detachably fitted into the injection hole (31c).

In one embodiment, the water supply member (30) includes a water supply tank (32a) and a distal tube portion (32b) connected to the water supply tank 32a, wherein the water supply tank (32a) and the distal tube portion 32b are connected through a connection tube (32a-1), wherein a coupler (32c) is installed at the proximal end of the distal tube portion (32b), wherein the coupler (32c) couples a sheet receiving tube portion 32d and the distal tube portion (32b) to each other, wherein the sheet module (20a) is inserted into the sheet receiving tube portion (32d), wherein a proximal wall (32e) is formed in the inner space within the sheet receiving tube portion (32d), wherein a water flow hole (32f) is formed in the proximal wall (32e) such that water is supplied to the sheet module (20a) through the water flow hole (32f).

In one embodiment, the water supply member (30) include a water supply bath (30a), and a fixing tube 30b in communication with the water supply bath (30a), wherein the sheet module (20) is inserted into the water supply bath (30a) through the fixing tube (32b).

In one embodiment, the sheet module 20 is disposed on the inner face of the mask 10 in a region of the nose and mouth of the face portion, wherein a shape-memory member 10a is provided on a top of a front face of the mask 10, wherein the loops 11 are disposed on both ends of the mask 10 respectively.

### Technical Effect

In accordance with the present invention, the multi-functional masking assembly having a humidity adjusting function may have the sheet module provided on the inner side surface of the mask worn to cover the nose and the mouth of the face, wherein the sheet module is located on the cheek parts of the face, and water is supplied to the sheet module so as to supply humidity to the nose and the mouth of the face and to adjust humidity around the nose and the mouth while the mask is worn, thereby: preventing a cold, dryness due to the air during air conditioning, and inhalation of polluted air, yellow dust, fine dust, and the like caused by air pollution while increasing respiratory health; and enabling people of all ages and genders to easily use the mask.

### DESCRIPTION OF THE DRAWINGS

FIG. 1 shows the first embodiment according to the present disclosure.

FIG. 2 is a schematic view of the water supply member of FIG. 1.

FIG. 3 shows the second embodiment according to the present disclosure.

FIG. 4 is a schematic view of the water supply member of FIG. 3.

FIG. 5 shows the third embodiment according to the present disclosure.

FIG. 6 shows the fourth embodiment according to the present disclosure.

FIG. 7 is a schematic view of a shape-memory member installed to a mask with a sheet module according to the present disclosure.

FIG. 8 is a schematic diagram showing an example of a mask according to the present disclosure.

### DETAILED DESCRIPTIONS

To fully understand the present disclosure, a preferred embodiment of the present disclosure is described with reference to the accompanying drawings.

The embodiments of the present disclosure may be embodied in many forms, and the scope of the present disclosure should not be construed as being limited to the embodiments detailed below. This embodiment is provided to further illustrate the present disclosure to those of ordinary skill in the art.

Therefore, the size or shape of the elements in the drawings and the like may be exaggerated to emphasize a clearer description. It should be noted that in the drawings, the same elements are denoted by the same reference numerals.

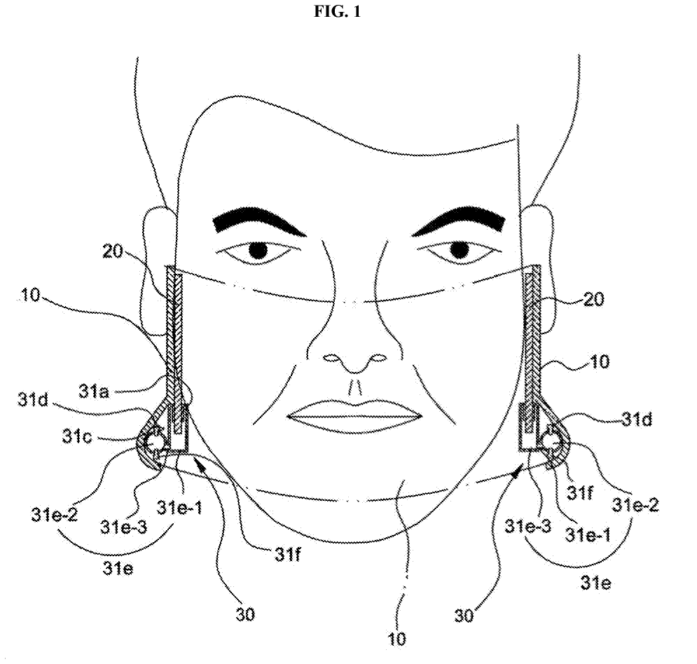

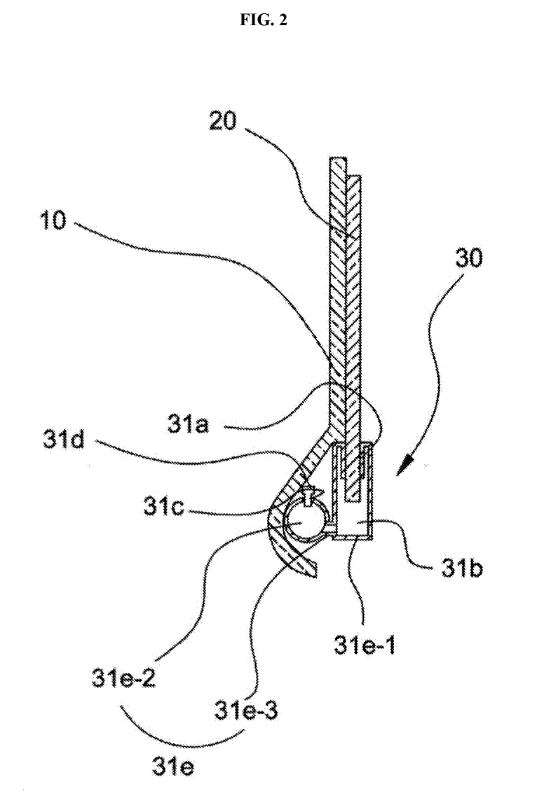

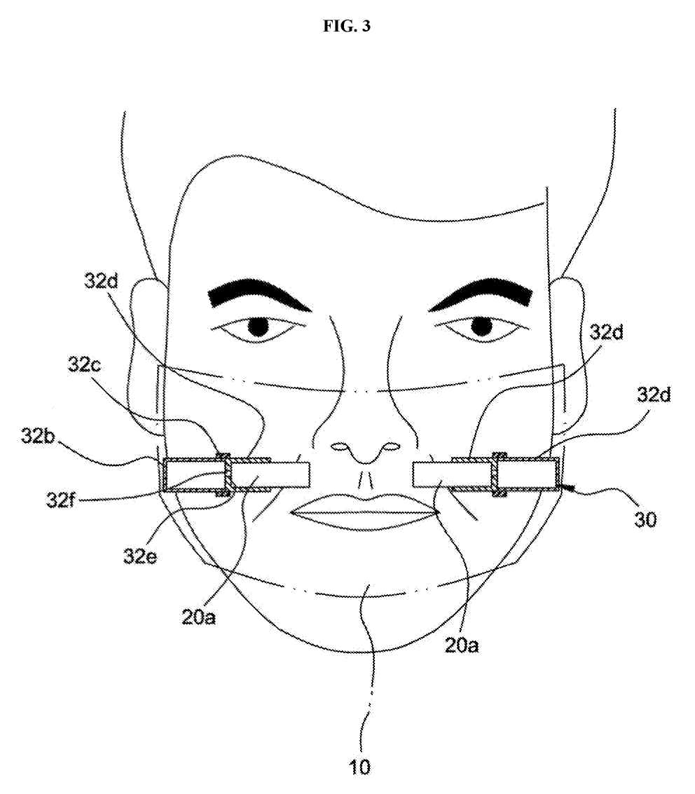

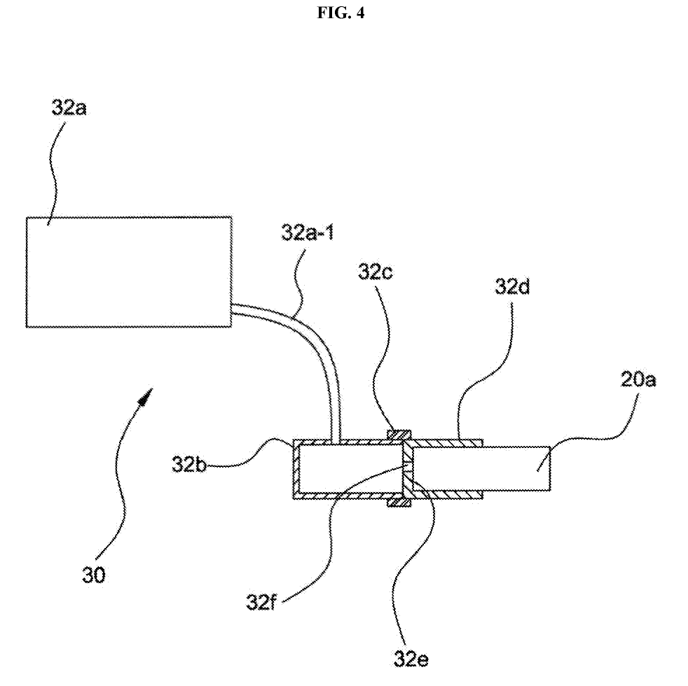

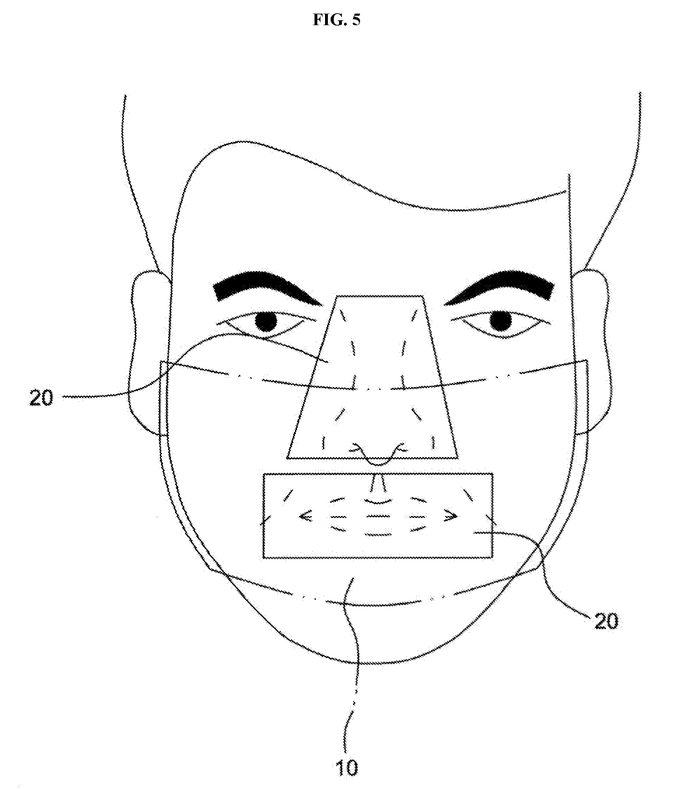

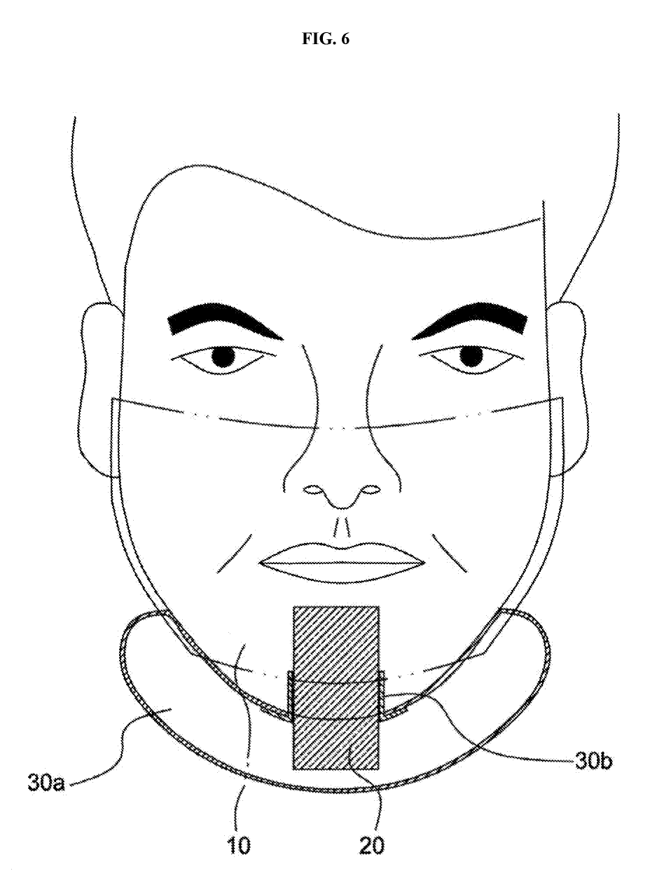

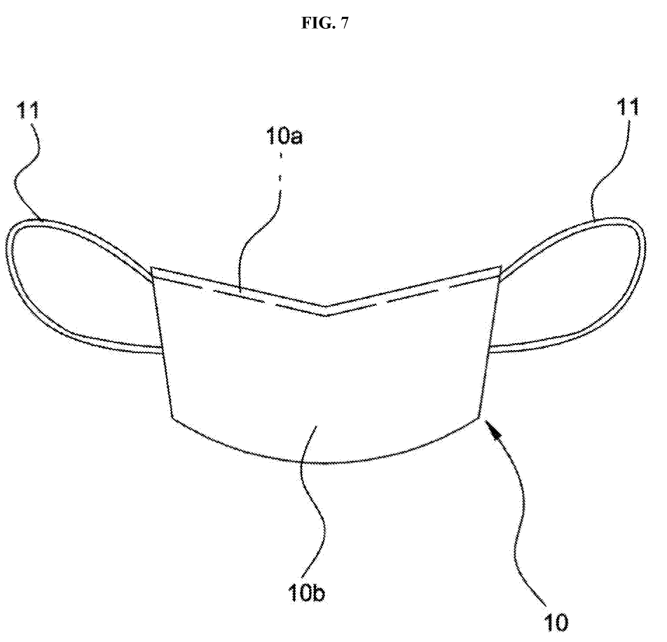

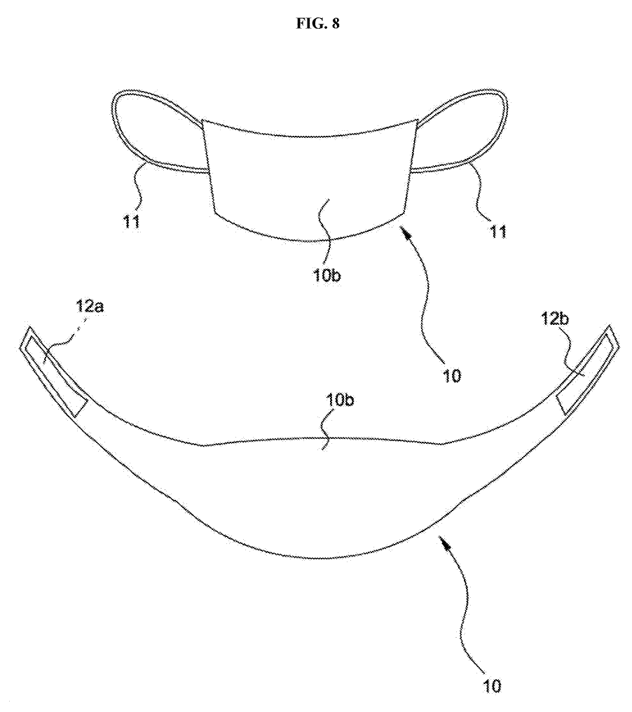

FIG. 1 shows the first embodiment according to the present disclosure. FIG. 2 is a schematic view of the water supply member of FIG. 1. FIG. 3 shows the second embodiment according to the present disclosure. FIG. 4 is a schematic view of the water supply member of FIG. 3. FIG. 5 shows the third embodiment according to the present disclosure. FIG. 6 shows the fourth embodiment according to the present disclosure. FIG. 7 is a schematic view of a shape-memory member installed to a mask with a sheet module according to the present disclosure. FIG. 8 is a schematic diagram showing an example of a mask according to the present disclosure.

A multi-functional masking assembly with humidity control according to the present disclosure will be described below with reference to FIGS. 1 to 8.

A multi-functional masking assembly with a humidity control function according to the present disclosure includes a mask 10, a sheet module 20, and a water supply member 30.

As shown in FIG. 8, the mask 10 includes a mask body 10b that may cover the nose and mouth of the face portion, and loops 11 that are mounted on both ends of the mask body 10b and are held on both ears of the user.

Alternatively, the mask 10 may include a female coupler member 12a mounted on one side of the mask body 10b that may cover the nose and mouth of the face portion, and a male coupler member 12b mounted on the other side of the mask body 10b and coupled to the female coupler member 12a.

The female and male coupler members 12a and 12b are preferably implemented using a Velcro type fastener.

The sheet module 20 may be positioned on the inner face of the mask 10 and on the cheek portion of the inner face of the mask 10, as shown in FIGS. 1 to 5.

Further, the sheet module 20 is preferably composed of cotton, charcoal, and sponge.

As described above, the sheet module 20 may be made up of cotton, charcoal, and sponge for the following reason: moisture absorption may be facilitated; the sheet module 20 may more easily filter pollutants such as dust and fine dust, pathogens of respiratory diseases resulting in colds and pneumonia, and airborne pathogens such as MERS (Middle East Respiratory Syndrome).

As shown in FIGS. 1 to 5, the water supply member 30 is configured to supply water to the sheet module 20.

The water supply member 30 as described above includes a water supply channel die, as shown in FIG. 1 and FIG. 2.

The lower end of the sheet module 20 is inserted into the inner space 31b in the vertical channel 31e-1 through the communication hole 31a defined in the vertical channel 31e-1.

The water supply channel 31e communicates with the inner space 31b defined in the vertical channel 31e-1 and supplies water into the inner space 31b. Thus, the lower end of the sheet module 20 absorbs water in the inner space 31b.

The water supply channel 31e includes a vertical channel 31e-1 having the communication hole 31a defined therein and the cylindrical channel 31e-2. The water supply channel 31e may further include a communication channel 31e-3 defined for connecting the vertical channel 31e-1 and the cylindrical channel 31e-2 to each other. External water is injected into the cylindrical channel 31e-2 through the injection hole sic formed in the cylindrical channel 31e-2.

The injected water is transferred into the vertical channel 31e-1 through the communication channel 31e-3.

The lower end of the sheet module 20 inserted into the internal space of the vertical channel 31e-1 through the communication hole 31a formed in the vertical channel 31e-1 absorbs water in the internal space of the vertical channel 31e-1. Thereby, water may be supplied to the nose and mouth of the face portion of the user, which achieves the humidity control function.

Further, the sheet module 20 is installed on the cheek portion of the face portion of the user. Water is supplied to the sheet module 20 installed on the cheek region, and humidity is controlled in the nose and mouth. External water is supplied into the inner space 31b through the injection hole 31c.

The plug 31d is detachably fitted into the injection hole 31c.

Further, a drain plug 31f is inserted into the hole defined in the lower end portion of the cylindrical channel 31e-2 so as to prevent the supplied water from overflowing and to allow water replacement for long use.

In another embodiment, the water supply member 30 includes a water supply tank 32a and a distal tube portion 32b connected to the water supply tank 32a, as shown in FIG. 3 and FIG. 4.

The water supply tank 32a and the distal tube portion 32b are connected through a connection tube 32a-1.

A coupler 32c is installed at the proximal end of the distal tube portion 32b.

The coupler 32c couples the sheet receiving tube portion 32d and the distal tube portion 32b to each other. The sheet module 20a is inserted into the sheet receiving tube portion 32d.

A proximal wall 32e is formed in the inner space within the sheet receiving tube portion 32d. A water flow hole 32f is formed in the proximal wall 32e. Water is supplied to the sheet module 20a through the water flow hole 32f.

The water supply member preferably has a discharging hole (not shown) and a filling hole (not shown) for filling water from the outside

Preferably, the sheet module 20a is disposed on the cheek of the face portion of the user and is adjacent the nose and the mouth.

In another embodiment, as shown in FIG. 6, the water supply member 30 is implemented as a water supply bath 30a.

The water supply bath 30a is mounted at the lower end of the jaw portion of the face portion.

There is a fixing tube 30b in communication with the water supply bath 30a.

The sheet module 20 is inserted into the water supply bath 30a through the fixing tube 32b.

The sheet module 20 is installed under the mouth of the face portion and is configured to supply moisture to the mouth and nose.

The sheet module 20 is wound in a circular shape. Thereby, the sheet module can be inserted into the fixing tube 32b formed in a cylindrical shape. Thereby, the sheet module is in contact with the water contained in the water supply bath 30a. With the capillary action of the sheet module 20, water rises from the bottom of the sheet module 20 to the top thereof. In this way, moisture is supplied to the mouth and nose of the face portion.

Further, the sheet module 20 is disposed on the inner face of the mask 10 in the region of the nose and mouth of the face portion, as shown in FIGS. 5 and 7.

The sheet module 20 absorbs water from the water supply member 30 such that humidity control may be done. Alternatively, the sheet module 20 in a dry state without water supply may be used.

A shape-memory member 10a may be provided on the upper portion of the front side of the mask 10 when loops 11 are formed on both ends of the mask 10.

By providing the shape-memory member 10a on top of the mask 10, the shape of the mask 10 may be matched to the shape of the nose and mouth of the face portion of the user.

In accordance with the present invention, the multi-functional masking assembly having a humidity adjusting function may have the sheet module 20 provided on the inner side surface of the mask 10 worn to cover the nose and the mouth of the face, wherein the sheet module 20 is located on the cheek parts of the face, and water is supplied to the sheet module so as to supply humidity to the nose and the mouth of the face and to adjust humidity around the nose and the mouth while the mask is worn, thereby: preventing a cold, dryness due to the air during air conditioning, and inhalation of polluted air, yellow dust, fine dust, and the like caused by air pollution while increasing respiratory health; and enabling people of all ages and genders to easily use the mask.

It will be appreciated that the embodiments according to the present disclosure described above are merely illustrative and that those skilled in the art will recognize that various modifications and equivalent embodiments are possible without departing from the scope of the present disclosure. It is therefore to be understood that the present disclosure is not limited solely to the form set forth in the foregoing description.

Accordingly, the true scope of protection of the present disclosure should be determined by the technical scope or spirit of the appended claims. Further, the present disclosure is to be understood as embracing the spirit of the present disclosure as defined by the appended claims, and all variations, equivalents, and alternatives falling within the scope of the present disclosure.

1. A multi-functional masking assembly with humidity control, the assembly comprising:
a mask having loops provided on both ends thereof and hung on the ears of an user, or female and male coupler members provided on both ends of the mask, wherein the female and male coupler members are removably coupled to each other;
a sheet module located on cheek portions of an inner face of the mask; and
a water supply member configured for supplying water to the sheet module.

2. The assembly of claim 1, wherein the sheet module is composed of cotton, charcoal, and sponge.

3. The assembly of claim 1, wherein the water supply member includes a water supply channel, wherein the water supply channel include a vertical channel, a cylindrical channel and a communication channel defined for connecting the vertical channel and the cylindrical channel to each other, wherein a lower end of the sheet module is inserted into an inner space defined in the vertical channel through an through-hole defined at a top portion of the vertical channel, wherein external water is injected into the cylindrical channel through an injection hole defined at the cylindrical channel, wherein the injected water is transferred into the vertical channel through the communication channel, wherein a plug is detachably fitted into the injection hole.

4. The assembly of claim 1, wherein the water supply member includes a water supply tank and a distal tube portion connected to the water supply tank, wherein the water supply tank and the distal tube portion are connected through a connection tube, wherein a coupler is installed at the proximal end of the distal tube portion, wherein the coupler couples a sheet receiving tube portion and the distal tube portion to each other, wherein the sheet module is inserted into the sheet receiving tube portion, wherein a proximal wall is formed in the inner space within the sheet receiving tube portion, wherein a water flow hole is formed in the proximal wall such that water is supplied to the sheet module through the water flow hole.

5. The assembly of claim 1, wherein the water supply member include a water supply bath, and a fixing tube in communication with the water supply bath, wherein the sheet module is inserted into the water supply bath through the fixing tube.

6. The assembly of claim 1, wherein the sheet module is disposed on the inner face of the mask in a region of the nose and mouth of the face portion, wherein a shape-memory member is provided on a top of a front face of the mask, wherein the loops are disposed on both ends of the mask respectively.
